(19) United States
(12) Patent Application Publication
(10) Pub. No.: US 2006/0062847 A1
(43) Pub. Date: Mar. 23, 2006

(54) Pharmaceutical dosage forms with impeded extractability of a sympathomimetic

(75) Inventors: Karl Kolter, Limburgerhof (DE); Marcus Wichtner, Waibstadt (DE); Kathrin Meyer, Feucht (DE); Heinz Einig, Neustadt (DE)

Correspondence Address: CONNOLLY BOVE LODGE & HUTZ, LLP, P O BOX 2207, WILMINGTON, DE, 19899, US

(73) Assignee: BASF AKTIENGESELLSCHAFT, Ludwigshafen (DE)

(21) Appl. No.: 11/225,176

(22) Filed: Sep. 14, 2005

(30) Foreign Application Priority Data
Sep. 15, 2004 (DE) 102004045037.4

(51) Int. Cl. A61K 9/20; C07C 67/36

(52) U.S. Cl. 424/464; 560/232

(57) ABSTRACT

A solid oral pharmaceutical dosage form of a sympathomimetic with impeded extractability of the sympathomimetic, comprising I) a sympathomimetic (component I) II) an excipient mixture (component II) composed of a) 5 to 50% by weight of hydroxyalkylcelluloses or alkylcelluloses or mixtures thereof b) 5 to 70% by weight of xanthan
c) 5 to 50% by weight of an alkaline earth metal salt of a C₁₀-C₃₀ fatty acid
d) 10 to 70% by weight of an alkali metal or alkaline earth metal salt of a mineral acid, of a sulfonic acid or of a C₂-C₆ carboxylic acid or mixtures thereof, where the total of components a) to d) is 100% by weight, where the ratio of active ingredient to excipient mixture II) is from 1:2 to 3:1 by weight.

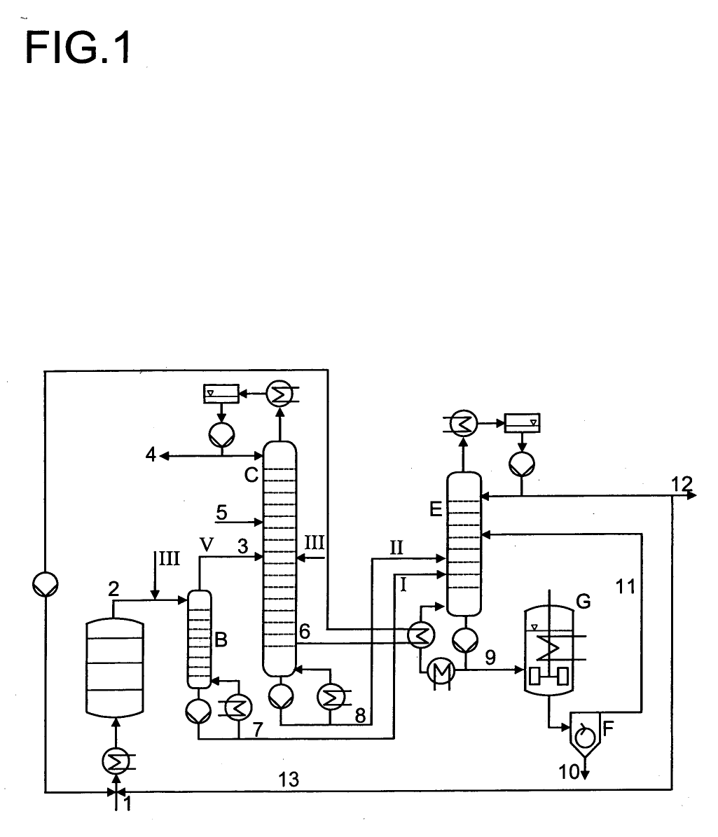

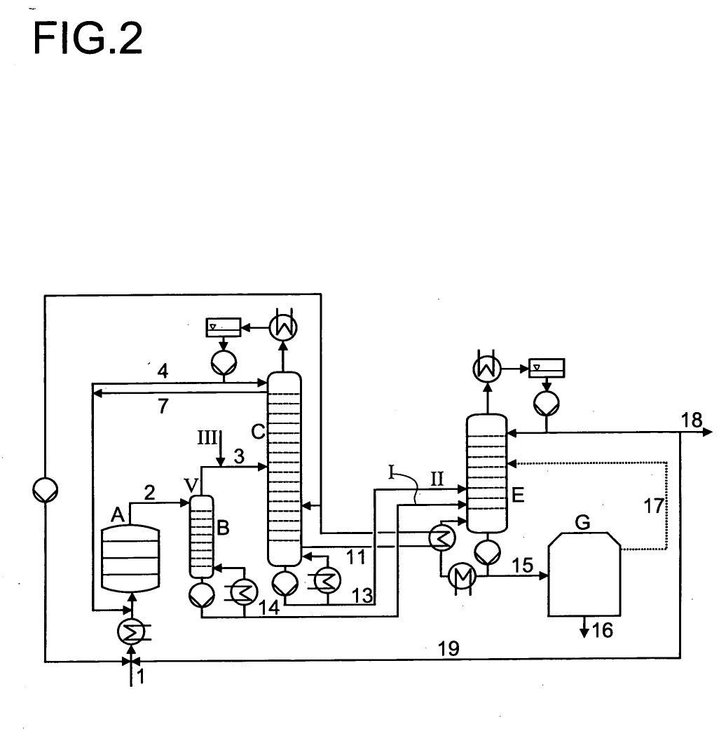

The present invention relates to improved pharmaceutical dosage forms to impede the extractability of a sympathomimetic from a pharmaceutical dosage form.

The amphetamines and ephedrones which lead to dependence are frequently employed in the drugs scene. They can for example be prepared in a relatively simple manner from the active ingredients ephedrine or pseudoephedrine, which are frequently employed against colds, or from phenylpropanolamines. Attempts are therefore frequently made to isolate the corresponding active ingredients from the medicines in order to process them further to the drugs.

Attempts have therefore been made to prevent the misuse of medicines through choice of the tablet ingredients.

WO 03/105808 describes a dosage form of opiates, opioids, tranquilizers and other narcotics which is secured against parenteral misuse by addition of a viscosity-increasing agent with the intention of preventing the possibility of parenteral administration of an aqueous extract of such dosage forms. The dosage forms described in this publication are preferably slow-release forms.

U.S. Pat. No. 6,197,314 states that the extraction of sympathomimetics is impeded through use of mixtures of pharmacologically suitable fats or gel formers and surfactants.

A combination of gel formers and surfactants is also employed according to U.S. Pat. No. 6,359,011 and U.S. Pat. No. 6,136,864 in order to prevent extraction of a sympathomimetic.

It has emerged, however, that prevention of extractability by the known processes still leaves room for improvements.

It is an object of the present invention to find pharmaceutical dosage forms with rapid release of the sympathomimetic which allow better prevention of the extractability of a sympathomimetic from the dosage form.

We have found that this object is achieved by pharmaceutical dosage forms which, besides a sympathomimetic (component I) comprise as component II an excipient mixture of

a. 5 to 50% by weight of a hydroxyalkylcellulose or of an alkylcellulose,

b. 5 to 70% by weight of xanthan

c. 5 to 50% by weight of an alkaline earth metal salt of a C₁₀-C₃₀ fatty acid, and

d. 10 to 70% by weight of an alkali metal or alkaline earth metal salt of a mineral acid, of a sulfonic acid or of a C₂-C₆ carboxylic acid, where the amounts of a) to d) add up to 100% by weight,

and, where appropriate, tableting excipients as component III.

Suitable sympathomimetics according to the invention are racemic ephedrine, (+)-ephedrine, (−)-ephedrine, racemic pseudoephedrine, (+)-pseudoephedrine, (−)-pseudoephedrine, and the corresponding phenylpropanolamines unsubstituted on the amino group, such as norephedrine or norpseudoephedrine. The active ingredients are normally in the form of their salts with physiologically tolerated acids, in particular as hydrochlorides or sulfates. The sympathomimetics are normally present in the dosage form in amounts of from 5 to 50% by weight, based on the total weight of the dosage form. Usual dosages for such active ingredients are 30 or 60 mg per dosage form.

The dosage forms may additionally comprise further active ingredients which are frequently combined with sympathomimetics. Such additional active ingredients which are suitable are: strong analgesics such as hydrocodone, dextromethorphan or codeine, weak analgesics such as acetaminophen or ibuprofen, antihistamines such as brompheniramine, chlorpheniramine or diphenhydramine.

The dosage forms comprise as component II a mixture of excipients a) to d) which are intended to prevent extractability of the sympathomimetic from the dosage form.

Suitable components a) are hydroxyalkylcelluloses such as hydroxypropylcellulose, hydroxyethylcellulose or hydroxypropylmethylcelluloses, alkylcelluloses such as methylcellulose or ethylcellulose. Hydroxypropylcellulose is preferably used. Excipients a) are preferably employed in amounts of from 7.5 to 40% by weight.

Xanthan is employed as excipient b). Xanthan is preferably used in amounts of from 10 to 60% by weight.

Suitable excipients c) are the alkaline earth metal salts of C₁₀-C₃₀ fatty acids. Magnesium stearate, calcium stearate or calcium behenate is preferably used. Ingredients c) are preferably used in amounts of from 7.5 to 40% by weight.

Suitable components d) are the alkali metal and alkaline earth metal salts of mineral acids, physiologically tolerated sulfonic acids or of C₂-C₆ carboxylic acids. Particularly suitable are sodium, potassium, magnesium and calcium salts. Mineral acids suitable for the purposes of this invention are hydrochloric acid, sulfuric acid, phosphoric acid and carbonic acid. Suitable salts are accordingly the chlorides, sulfates, hydrogensulfates, phosphates, hydrogenphosphates, dihydrogenphosphates, carbonates and hydrogencarbonates. Suitable sulfonic acids are in particular methanesulfonic acid, toluenesulfonic acid, benzenesulfonic acid. Suitable salts of organic C₂-C₆ carboxylic acids are the corresponding salts of lactic acid, tartaric acid, acetic acid, malonic acid, fumaric acid, maleic acid, succinic acid, citric acid. These excipients d) are preferably employed in amounts of from 20 to 70% by weight.

The data in % by weight for excipients a) to d) are based on the total amount of the excipient mixture used as component II, with the amounts of a) to d) adding up to 100% by weight.

The proportion of component II in the dosage form is chosen so that the ratio of sympathomimetic to component II is from 1:2 to 3:1, preferably 1:1.5 to 2:1, by weight. The proportion of component II in the total weight of the dosage form may be from 7.5 to 60, preferably 10 to 50, % by weight.

The dosage forms may additionally comprise conventional tableting aids such as fillers, disintegrants, lubricants, flow regulators or binders.

Suitable fillers are lactose, starches, mannitol, sorbitol, xylitol, calcium hydrogenphosphate or microcrystalline cellulose.

The amount of filler can be adjusted depending on the desired total weight of the dosage form. It is normally in the range from 0 to 60% by weight based on the total weight of the dosage form.

Suitable disintegrants are crospovidone, croscarmellose or crosslinked sodium carboxymethylstarch. The disintegrants are preferably employed in amounts of from 2 to 20% by weight based on the total weight of the dosage form.

It is also possible to add if desired as tableting aid a small amount of a further binder such as, for example, polyvinylpyrrolidone to the filler.

The use of basic polymers such as, for example, aminoalkyl methacrylate copolymer type E, JP in amounts of from 2 to 20% by weight based on the total weight of the dosage form is also possible.

It is additionally possible to add to the tableting excipients also pharmaceutically tolerated substances such as aromas, flavor improvers or antioxidants in the amounts customary for this purpose. It is also possible where appropriate to add in addition lubricants and flow regulators.

Release from the dosage forms of the invention is intended to be fast. Fast release means that at least 80% of the sympathomimetic are released after one hour. The release can be determined with the aid of the paddle method of the European Pharmacopoeia (PhEur 4.8). The dosage forms additionally disintegrate rapidly, i.e. the dosage form has completely disintegrated after one hour, preferably after 30 minutes.

The dosage form of the invention can be produced by processing all the ingredients in a conventional way to a physical mixture and then compressing to tablets. It is also possible for the excipient mixture for impeding extractability (component II) initially to be processed to a premix, and this premix then to be mixed with the other ingredients.

Such a premix can also be produced as granules by conventional granulation techniques or be pelleted to give pellets. Dry granulation using a roller compactor is also possible.

A further possibility is for this premix to be coated to delay onset of swelling in water. The rate of disintegration and of release of active ingredient is further increased in this way. Suitable coating materials are film formers which are resistant to gastric juice or slow release, such as, for example, methacrylic acid-ethyl acrylate (1:1) copolymer, polyvinyl acetate, cellulose acetate phthalate, hydroxypropylmethylcelluose acetate phthalate.

Preferred dosage forms are according to the invention tablets or film-coated tablets. In a particular embodiment, multilayer tablets can be produced, with one layer comprising component I and the other comprising component II. The further conventional tableting excipients can if required be distributed over the two layers.

In a further embodiment, however, the ingredients can also be packed in a capsule.

The extraction methods described below may be applied for example in the improper extraction of the sympathomimetic and can be used to check the impeding of extractability.

### Method A

100 tablets are crushed with a mortar and pestle. An aqueous solution is prepared from the crushed product by adding 100 ml of water and stirring with a magnetic stirrer. The active ingredient base is precipitated by adjusting the pH to above 12 by adding sodium hydroxide solution. The active ingredient base is extracted by adding 100 ml of organic solvent (e.g. dichloromethane, ethyl acetate, toluene) and stirring with the magnetic stirrer for 2 h. The two-phase mixture is left to stand for at least 24 h for phase separation. The phase floating on top is decanted off. The active ingredient base is crystallized by evaporating the organic solvent from the organic phase.

### Method B

1000 tablets are crushed with a mortar and pestle. The lipophilic constituents of the tablet are removed from the crushed product by prewashing with toluene. A vacuum filtration is then carried out with a filter of pore size below 25 μm. The active ingredient salt is extracted from the filtration residue by adding 350 ml of water and stirring with a magnetic stirrer for 1 h. The hydrophilic constituents are filtered off from the active ingredient salt, which is dissolved in the aqueous phase, by vacuum filtration (pore size below 25 μm). An aqueous solution of active ingredient salt is obtained by two repetitions of the one-phase aqueous extraction and removal of the hydrophilic tablet constituents by vacuum filtration. After addition of a few drops of hydrochloric acid, the filtrate obtained from the three vacuum filtrations is evaporated to about 200 ml. After the enriched aqueous active ingredient salt solution has cooled to room temperature, the pH can be adjusted to above 12 by adding sodium hydroxide solution, and thus the active ingredient base be precipitated. The active ingredient base is extracted by subsequent addition of 100 ml of organic solvent (e.g. dichloromethane, ethyl acetate, toluene) and stirring with the magnetic stirrer for 2 h. The two-phase mixture is left to stand for at least 24 h for phase separation. The phase floating on top is decanted off. The active ingredient base is crystallized by evaporating the organic solvent from the organic phase.

Note: Method B analogous to [Uncle Fester/Secrets of Methamphetamine Manufacture/page 155 ff/1997]

It has emerged that extraction with these methods is no longer possible in the case of the dosage forms of the invention. On addition of an organic solvent (method A), stable emulsions are formed and the active ingredient-comprising organic phase cannot be removed from them. No filtration is possible on application of method B.

The dosage forms obtained according to the invention are therefore protected against misuse through the impeding or prevention of the extractability of the sympathomimetic. Surprisingly, however, the fast release which is desired for such active ingredients on proper use, and the rapid disintegration of the tablet, are not impaired through use of the excipient mixture (component II) impeding the extractability of the active ingredient.

### EXAMPLES

### Example A

Composition

| | |
|---|---|
| Pseudoephedrine HCl | 30.0 mg |
| HPMC | 4.0 mg |
| (Methocel K100M Premium) | |
| Xanthan | 12.0 mg |
| (Xanthural 75) | |
| Magnesium stearate | 4.0 mg |
| Sodium chloride | 10.0 mg |
| Ludipress¹⁾ | 140.0 mg |
| Σ | 200.0 mg |

¹⁾Mixture of 93% by weight of lactose monohydrate, 3.5% by weight of povidone K30, 3.5% by weight of crospovidone

Tablet Production

The ingredients hydroxypropylmethylcellulose, xanthan, magnesium stearate and sodium chloride were mixed in a free-fall mixer for 10 min. The active ingredient was mixed with this mixture and the tableting excipient Ludipress in a free-fall mixer for a further 10 min and then compressed in a rotary press (Korsch PH 100/6) to tablets with a weight of 200 mg and a diameter of 8.0 mm under a compressive force of 25 kN.

### Example B

Composition

| | |
|---|---|
| Pseudoephedrine HCl | 30.0 mg |
| HPC | 12.0 mg |
| (Klucel HF) | |
| Xanthan | 6.0 mg |
| (Xanthural 75) | |
| Magnesium stearate | 6.0 mg |
| Potassium chloride | 6.0 mg |
| Kollidon CL | 20.0 mg |
| MCC | 120.0 mg |
| (Avicel PH-102)²⁾ | |
| Σ | 200.0 mg |

²⁾Microcrystalline cellulose with a particle size of approx. 100 μm.

Tablet Production

The ingredients hydroxypropylcellulose, xanthan, magnesium stearate and potassium chloride were mixed in a free-fall mixer for 10 min. The active ingredient was mixed with this mixture and the tableting excipients Kollidon CL and Avicel PH-102 in a free-fall mixer for a further 10 min and then compressed in a rotary press (Korsch PH 100/6) to tablets with a weight of 200 mg and a diameter of 8.0 mm under a compressive force of 25 kN.

### Example C

Composition

| | |
|---|---|
| Pseudoephedrine HCl | 30.0 mg |
| HPC | 5.0 mg |
| (Klucel HF) | |
| Xanthan | 5.0 mg |
| (Xanthural 75) | |
| Magnesium stearate | 15.0 mg |
| Sodium chloride | 5.0 mg |
| Ludipress | 140.0 mg |
| Σ | 200.0 mg |

Tablet Production

The ingredients hydroxypropylcellulose, xanthan, magnesium stearate and sodium chloride were mixed in a free-fall mixer for 10 min. The active ingredient was mixed with this mixture and the tableting excipient Ludipress in a free-fall mixer for a further 10 min and then compressed in a rotary press (Korsch PH 100/6) to tablets with a weight of 200 mg and a diameter of 8.0 mm under a compressive force of 25 kN.

### Example D

Composition

| | |
|---|---|
| Pseudoephedrine HCl | 60.0 mg |
| HPMC | 3.0 mg |
| (Methocel K100M Premium) | |
| Xanthan | 18.0 mg |
| (Xanthural 75) | |
| Magnesium stearate | 3.0 mg |
| Sodium chloride | 36.0 mg |
| Kollidon 30 | 6 mg |
| Ludipress | 80.0 mg |
| Σ | 206.0 mg |

Tablet Production

The ingredients hydroxypropylmethylcellulose, xanthan and sodium chloride were mixed in a free-fall mixer for 10 min. This mixture was granulated with 10 percent ethanolic Kollidon 30 solution. The granules were mixed with magnesium stearate, the active ingredient and the tableting excipient Ludipress in a free-fall mixer for a further 10 min and then compressed in a rotary press (Korsch PH 100/6) to tablets with a weight of 206 mg and a diameter of 8.0 mm under a compressive force of 25 kN.

### Example E

Composition

| | |
|---|---|
| Pseudoephedrine HCl | 60.0 mg |
| HPC | 9.0 mg |
| (Klucel HF) | |
| Xanthan | 30.0 mg |
| (Xanthural 75) | |
| Magnesium stearate | 6.0 mg |
| Potassium chloride | 18.0 mg |
| Kollidon CL | 25.0 mg |
| Ludipress | 105.0 mg |
| Σ | 253.0 mg |

Tablet Production

The ingredients hydroxypropylcellulose, xanthan, magnesium stearate and potassium chloride were mixed in a free-fall mixer for 10 min (mixture I). The active ingredient was mixed with the tableting excipients Kollidon CL and Ludipress in a free-fall mixer for 10 min (mixture II). Bilayer tablets with a weight of 253 mg and a diameter of 8.0 mm, where the lower layer consisted of 190 mg of mixture II and the upper of 63 mg of mixture I, were compressed in a rotary press under a compressive force of 25 kN.

### Example F

Composition

| | |
|---|---|
| Pseudoephedrine HCl | 60.0 mg |
| HPMC | 15.0 mg |
| (Methocel K100M Premium) | |
| Xanthan | 7.5 mg |
| (Xanthural 75) | |
| Magnesium stearate | 5.0 mg |
| Potassium chloride | 2.5 mg |
| Kollidon CL | 20.0 mg |
| MCC | 70.0 mg |
| (Avicel PH-102) | |
| CaHPO₄ * 2H₂O | 70.0 mg |
| (Di-Tab) | |
| Σ | 250.0 mg |

Tablet Production

The ingredients hydroxypropylmethylcellulose, xanthan, magnesium stearate and potassium chloride were mixed in a free-fall mixer for 10 min. The active ingredient was mixed with this mixture and the tableting excipients Kollidon CL, Avicel PH-102 and Di-Tab in a free-fall mixer for a further 10 min and then compressed in a rotary press (Korsch PH 100/6) to tablets with a weight of 250 mg and a diameter of 8.0 mm under a compressive force of 25 kN.

### Example G

Composition

| | |
|---|---|
| Pseudoephedrine HCl | 60.0 mg |
| HPMC | 6.0 mg |
| (Methocel K100M Premium) | |
| Xanthan | 18.0 mg |
| (Xanthural 75) | |
| Magnesium stearate | 6.0 mg |
| Sodium chloride | 30.0 mg |
| MCC | 65.0 mg |
| (Avicel PH-102) | |
| CaHPO₄ * 2H₂O | 65.0 mg |
| (Di-Tab) | |
| Σ | 250.0 mg |

Tablet Production

The ingredients hydroxypropylmethylcellulose, xanthan, magnesium stearate and potassium chloride were mixed in a free-fall mixer for 10 min. The active ingredient was mixed with this mixture and the tableting excipients Avicel PH-102 and Di-Tab in a free-fall mixer for a further 10 min and then compressed in a rotary press (Korsch PH 100/6) to tablets with a weight of 250 mg and a diameter of 8.0 mm under a compressive force of 25 kN.

Example H

Composition

| | |
|---|---|
| Pseudoephedrine HCl | 60.0 mg |
| HPC | 15.0 mg |
| (Klucel HF) | |
| Xanthan | 12.0 mg |
| (Xanthural 75) | |
| Magnesium stearate | 6.0 mg |
| Sodium chloride | 27.0 mg |
| MCC | 65.0 mg |
| (Avicel PH-102) | |
| CaHPO₄ * 2H₂O | 65.0 mg |
| (Di-Tab) | |
| Σ | 250.0 mg |

Tablet Production

The ingredients hydroxypropylcellulose, xanthan, magnesium stearate and sodium chloride were mixed in a free-fall mixer for 10 min. The active ingredient was mixed with this mixture and the tableting excipients Avicel PH-102 and Di-Tab in a free-fall mixer for a further 10 min and then compressed in a rotary press (Korsch PH 100/6) to tablets with a weight of 250 mg and a diameter of 8.0 mm under a compressive force of 25 kN.

### Example J

Composition

| | |
|---|---|
| Pseudoephedrine HCl | 60.0 mg |
| HPC | 6.0 mg |
| (Klucel HF) | |
| Xanthan | 18.0 mg |
| (Xanthural 75) | |
| Magnesium stearate | 12.0 mg |
| Sodium chloride | 24.0 mg |
| Kollidon CL | 25.0 mg |
| MCC | 52.5 mg |
| (Avicel PH-102) | |
| CaHPO₄ * 2H₂O | 52.5 mg |
| (Di-Tab) | |
| Σ | 250.0 mg |

Tablet Production

The ingredients hydroxypropylcellulose, xanthan, magnesium stearate and potassium chloride were mixed in a free-fall mixer for 10 min. The active ingredient was mixed with this mixture and the tableting excipients Kollidon CL, Avicel PH-102 and Di-Tab in a free-fall mixer for a further 10 min and then compressed in a rotary press (Korsch PH 100/6) to tablets with a weight of 250 mg and a diameter of 8.0 mm under a compressive force of 25 kN.

### Example K

Composition

| | |
|---|---|
| Pseudoephedrine HCl | 60.0 mg |
| HPMC | 3.0 mg |
| (Methocel K100M Premium) | |
| Xanthan | 30.0 mg |
| (Xanthural 75) | |
| Magnesium stearate | 15.0 mg |
| Potassium chloride | 12.0 mg |
| Kollidon CL | 30.0 mg |
| Ludipress | 100.0 mg |
| Σ | 250.0 mg |

Tablet Production

The ingredients hydroxypropylcellulose, xanthan, magnesium stearate and potassium chloride were mixed in a free-fall mixer for 10 min. The active ingredient was mixed with this mixture and the tableting excipients Kollidon CL and Ludipress in a free-fall mixer for a further 10 min and then compressed in a rotary press (Korsch PH 100/6) to tablets with a weight of 250 mg and a diameter of 8.0 mm under a compressive force of 25 kN.

### Example L

Composition

| | |
|---|---|
| Pseudoephedrine HCl | 60.0 mg |
| HPC | 6.0 mg |
| (Klucel HF) | |
| Xanthan | 18.0 mg |
| (Xanthural 75) | |
| Magnesium stearate | 6.0 mg |
| Sodium chloride | 24.0 mg |
| Potassium chloride | 24.0 mg |
| MCC | 81.0 mg |
| (Avicel PH-102) | |
| CaHPO₄ * 2H₂O | 81.0 mg |
| (Di-Tab) | |
| Σ | 300.0 mg |

Tablet Production

The ingredients hydroxypropylcellulose, xanthan, sodium chloride and potassium chloride were mixed in a free-fall mixer for 10 min. This mixture was compacted in a roller compactor. The compact was passed through a sieve with a mesh width of 1.2 mm and mixed with magnesium stearate, the active ingredient and the tableting excipients Avicel PH-102 and Di-Tab for in a free-fall mixer for 10 min, and then compressed in a rotary press (Korsch PH 100/6) to tablets with a weight of 300 mg and a diameter of 10.0 mm under a compressive force of 25 kN.

### Example M

Composition

| | |
|---|---|
| Pseudoephedrine HCl | 60.0 mg |
| HPMC | 6.0 mg |
| (Methocel K100M Premium) | |
| Xanthan | 18.0 mg |
| (Xanthural 75) | |
| Magnesium stearate | 6.0 mg |
| Sodium chloride | 12.0 mg |
| MCC | 99.0 mg |
| (Avicel PH-102) | |
| CaHPO₄ * 2H₂O | 99.0 mg |
| (Di-Tab) | |
| Σ | 300.0 mg |

Tablet Production

The ingredients hydroxypropylmethylcellulose, xanthan, magnesium stearate and NaCl were mixed in a free-fall mixer for 10 min. This mixture was mixed with the tableting excipients Avicel PH-102 and Di-Tab in a free-fall mixer for a further 10 min and then compressed in a rotary press (Korsch PH 100/6) to tablets with a weight of 300 mg and a diameter of 10.0 mm under a compressive force of 25 kN.

The extractability was determined by processing the tablets obtained as described in Examples A to M in accordance with methods A and B. It emerged that extraction of the active ingredient was not possible in this way.

| Formula | | |
|---|---|---|
| Example | Extraction by method A | Extraction by method B |
| A | highly viscous emulsion, stable | no filtration possible |
| | for >2 weeks | |
| B | highly viscous emulsion, stable | no filtration possible |
| | for >2 weeks | |
| C | highly viscous emulsion, stable | no filtration possible |
| | for >2 weeks | |
| D | highly viscous emulsion, stable | no filtration possible |
| | for >2 weeks | |
| E | highly viscous emulsion, stable | no filtration possible |
| | for >2 weeks | |
| F | highly viscous emulsion, stable | no filtration possible |
| | for >2 weeks | |
| G | highly viscous emulsion, stable | no filtration possible |
| | for >2 weeks | |
| H | highly viscous emulsion, stable | no filtration possible |
| | for >2 weeks | |
| J | highly viscous emulsion, stable | no filtration possible |
| | for >2 weeks | |
| K | highly viscous emulsion, stable | no filtration possible |
| | for >2 weeks | |
| L | highly viscous emulsion, stable | no filtration possible |
| | for >2 weeks | |
| M | highly viscous emulsion, stable | no filtration possible |
| | for >2 weeks | |

Tablet Disintegration and Release

| Formula | | |
|---|---|---|
| Example | Tablet disintegration | Release |
| A | <30 min | 80% after 30 min |
| D | <60 min | 80% after 60 min |
| E | <60 min | 80% after 15 min |
| J | <60 min | 80% after 60 min |
| L | <60 min | 80% after 60 min |
| M | <60 min | 80% after 60 min |

The disintegration time is measured by the method of the German Pharmacopoeia 10, V.5.1.

Release

The active ingredient release rate is determined by the method of the European Pharmacopoeia (PhEur 4.8).

We claim:

1. A solid oral pharmaceutical dosage form of a sympathomimetic with impeded extractability of the sympathomimetic, comprising
the sympathomimetic (component I) and
an excipient mixture (component II) consisting of
a) 5 to 50% by weight of a hydroxyalkylcellulose or an alkylcellulose or mixtures thereof
b) 5 to 70% by weight of xanthan
c) 5 to 50% by weight of an alkaline earth metal salt of a C₁₀-C₃₀ fatty acid
d) 10 to 70% by weight of an alkali metal or alkaline earth metal salt of a mineral acid, of a sulfonic acid or of a C₂-C₆ carboxylic acid or mixtures thereof,
where the total of components a) to d) is 100% by weight, and
where the ratio of the sympathomimetic to the excipient mixture is from 1:2 to 3:1 by weight.

2. The dosage form according to claim 1, further comprising tableting aids as component III.

3. The dosage form according to claim 1 wherein the hydroxyalkylcellulose or alkylcellulose is selected from the group consisting of hydroxypropylcellulose, hydroxyethylcellulose, hydroxypropylmethylcellulose, methylcellulose, ethylcellulose and mixtures thereof.

4. The dosage form according to claim 1 wherein the alkaline earth metal salt fatty acid includes magnesium stearate.

5. The dosage form according to claim lwherein the mineral acid, the sulfuric acid or the carboxylic acid is selected from the group consisting of hydrochloric acid, sulfuric acid, phosphoric acid, carbonic acid, methanesulfonic acid, acetic acid, lactic acid, tartaric acid, malonic acid, fumaric acid, maleic acid, succinic acid, citric acid and mixtures thereof.

6. The dosage form according to claim 1, wherein the excipient mixture comprises
a) 7.5 to 40% by weight of hydroxyalkylcelluloses or alkylcelluloses or mixtures thereof
b) 10 to 60% by weight of xanthan
c) 7.5 to 40% by weight of an alkaline earth metal salt of a C₁₀-C₃₀ fatty acid
d) 20 to 70% by weight of an alkali metal or alkaline earth metal salt of a mineral acid, of a sulfonic acid or of a C₂-C₆ carboxylic acid and mixtures thereof.

7. The dosage form according to claim 1 further comprising at least one tableting excipient selected from the group consisting of fillers, disintegrants, lubricants, flow regulators, binders, aromas, flavor improvers and antioxidants.

8. The dosage form according to claim 1 further comprising at least one filler ingredient selected from the group consisting of lactose, starch, mannitol, sorbitol, xylitol, calcium hydrogen phosphate and microcrystalline cellulose.

9. The dosage form according to claim 1 further comprising at least one disintegrant selected from the group of crospovidone, croscarmellose and crosslinked sodium carboxymethylstarch.

10. The use of an excipient mixture consisting of
a) 5 to 50% by weight of hydroxyalkylcelluloses or alkylcelluloses or mixtures thereof
b) 4 to 70% by weight of xanthan
c) 5 to 50% by weight of an alkaline earth metal salt of a C₁₀-C₃₀ fatty acid
d) 20 to 70% by weight of an alkali metal or alkaline earth metal salt of a mineral acid, of a sulfonic acid or of a C₂-C₆ carboxylic acid or mixtures thereof
where the total of components a) to d) is 100% by weight,
for impeding the extractability of a sympathomimetic from a pharmaceutical dosage form.

11. The dosage form according to claim 1 wherein the sympathomimetic is selected from the group consisting of racemic ephedrine, (+)-ephedrine, (−)-ephedrine, racemic pseudoephedrine, (+)-pseudoephedrine, (−)-pseudoephedrine, and the corresponding phenylpropanolamines unsubstituted on the amino group which includes norephedrine or norpseudoephedrine.

12. The dosage form according to claim 11 wherein the sympathomimetic is present from 30 mg to 60 mg.

13. The dosage form according to claim 1 wherein the sympathomimetic and xanthan are present as a weight ratio of sympathomimetic:xanthan from 2:1 to 8:1.

14. The dosage form according to claim 13 wherein the weight ratio is from 2:1 to 5:1.

15. A solid oral pharmaceutical dosage form comprising:
a sympathomimetic; and
an excipient mixture consisting essentially of
5 to 50% by weight of a hydroxyalkylcellulose or an alkylcellulose or mixtures thereof,
5 to 70% by weight of xanthan,
5 to 50% by weight of an alkaline earth metal salt of a C₁₀-C₃₀ fatty acid, and
10 to 70% by weight of an alkali metal or alkaline earth metal salt of a mineral acid, a sulfonic acid, a C₂-C₆ carboxylic acid or mixtures thereof, wherein the sympathomimetic and excipient mixture are present as a weight ratio of sympathomimetic:excipient mixture from 1:2 to 3:1.

16. The dosage form according to claim 15 wherein the hydroxyalkylcellulose or alkylcellulose is selected from the group consisting of hydroxypropylcellulose, hydroxyethylcellulose, hydroxypropylmethylcellulose, methylcellulose, ethylcellulose and mixtures thereof.

17. The dosage form according to claim 15 wherein the alkaline earth metal salt fatty acid includes magnesium stearate.

18. The dosage form according to claim 15, wherein the excipient mixture comprises:
7.5 to 40% by weight of the hydroxyalkylcellulose or the alkylcellulose or mixture thereof,
10 to 60% by weight of the xanthan,
7.5 to 40% by weight of the alkaline earth metal salt of a C₁₀-C₃₀ fatty acid, and
20 to 70% by weight of the alkali metal or alkaline earth metal salt of a mineral acid, of a sulfonic acid or of a C₂-C₆ carboxylic acid or mixture thereof.

19. The dosage form according to claim 15 wherein the sympathomimetic and xanthan are present as a weight ratio of sympathomimetic:xanthan from 2:1 to 8:1.

20. The dosage form according to claim 15 wherein the sympathomimetic is selected from the group consisting of racemic ephedrine, (+)-ephedrine, (−)-ephedrine, racemic pseudoephedrine, (+)-pseudoephedrine, (−)-pseudoephedrine, and the corresponding phenylpropanolamines unsubstituted on the amino group which includes norephedrine or norpseudoephedrine.
